(19) United States
(12) Patent Application Publication
(10) Pub. No.: US 2022/0196770 A1
(43) Pub. Date: Jun. 23, 2022

(54) METHOD FOR EXECUTING AT LEAST ONE APPLICATION ON A MAGNETIC RESONANCE DEVICE

(71) Applicant: Siemens Healthcare GmbH, Erlangen (DE)

(72) Inventors: Christoph Forman, Uttenreuth (DE); Peter Gall, Uttenreuth (DE); Uvo Hölscher, Erlangen (DE)

(73) Assignee: Siemens Healthcare GmbH, Erlangen (DE)

(21) Appl. No.: 17/558,946

(22) Filed: Dec. 22, 2021

(30) Foreign Application Priority Data
Dec. 22, 2020 (EP) 20216753.2

(51) Int. Cl. G01R 33/54

(52) U.S. Cl. CPC G01R 33/543; G01R 33/546

(57) ABSTRACT

In a method for executing at least one application on a magnetic resonance (MR) device, the at least one application is stored on a separately implemented memory, a communication and/or data exchange is performed with the at least one application using an interface of the MR device, at least one item of hardware information relating to the MR device is communicated to the at least one application using an information channel of the interface, at least one parameter of the at least one application is matched to the at least one item of hardware information of the MR device, and at least one item of control information is provided using a control channel of the interface, by the at least one application of the magnetic resonance device, to execute the at least one application.

### CROSS REFERENCE TO RELATED APPLICATIONS

This patent application claims priority to European Patent Application No. 20216753.2, filed Dec. 22, 2020, which is incorporated herein by reference in its entirety.

### BACKGROUND

With many magnetic resonance (MR) devices, replacing software often involves reinstalling the entire software. In addition, the software is specifically tailored to the magnetic resonance device, in particular to the hardware properties of the magnetic resonance device. Such hardware properties include a magnetic field strength and/or a maximum gradient field strength and/or a design of a receiving device of a radiofrequency system, etc. However, this means that replacing the software is very elaborate and therefore usually occurs only every few years. Replacing individual software components for a magnetic resonance device would therefore also invariably result in a complete software reinstallation, which is, however, very elaborate. Consequently, the individual software components are compiled and are not incorporated until the next reinstallation. The software components are therefore only available for use after this reinstallation of the entire software.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 schematically illustrates a system including a magnetic resonance device and an application, according to an exemplary embodiment of the disclosure.

FIG. 2 schematically illustrates a system including a magnetic resonance device and an application according to an exemplary embodiment of the disclosure.

FIG. 3 schematically illustrates a system including a magnetic resonance device and an application according to an exemplary embodiment of the disclosure.

FIG. 4 shows an application structure of an application according to an exemplary embodiment of the disclosure.

FIG. 5 is a flowchart of a method for executing at least one application on a magnetic resonance device according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to enable individual software components to be used independently of a software installation.

The disclosure proceeds from a method for executing at least one application on a magnetic resonance device, wherein:
the at least one application is stored on a separately implemented memory,
the magnetic resonance device comprises an interface by means of which communication and/or data exchange with the at least one application takes place,
at least one item of hardware information relating to the magnetic resonance device is transmitted to the at least one application by means of an information channel of the interface,
at least one parameter of the at least one application is aligned to the at least one item of hardware information relating to the magnetic resonance device, and
the at least one application is executed, wherein, by means of a control channel of the interface, at least one item of control information is provided by the at least one application of the magnetic resonance device for executing the at least one application.

The magnetic resonance device preferably comprises a medical and/or diagnostic magnetic resonance device which is designed and/or configured to acquire medical and/or diagnostic image data, in particular medical and/or diagnostic magnetic resonance image data, of a patient. The magnetic resonance device comprises a scanner for this purpose. The scanner of the magnetic resonance device preferably comprises a detector, in particular a magnet unit, for detecting the medical and/or diagnostic image data. Said scanner, in particular the magnet unit, preferably comprises a main magnet, a gradient system and a radiofrequency antenna. The radiofrequency antenna is fixed within the scanner and is designed and/or configured to emit an excitation pulse. The magnetic resonance device also comprises at least one local radiofrequency coil designed to receive a magnetic resonance signal. For this purpose, the local radiofrequency coil is disposed and/or arranged around the region of the patient to be examined. Individual local radiofrequency coils are preferably specifically designed for an examination region of the patient, such as a radiofrequency head coil or a radiofrequency knee coil, etc.

The main magnet is designed to generate a homogeneous main magnetic field with a defined and/or specific magnetic field strength, such as a defined and/or specific magnetic field strength of 3 T or 1.5 T, etc. In particular, the main magnet is designed to generate a powerful, constant and homogeneous main magnetic field. The homogeneous main magnetic field is preferably located and/or available within a patient receiving region of the magnetic resonance device. The gradient system is designed to generate magnetic field gradients used for spatial encoding during imaging.

The patient receiving region is designed and/or constructed to accommodate the patient, in particular the region of the patient to be examined, for a medical magnetic resonance examination. For this purpose, for example, the patient receiving region is cylindrical and/or cylindrically enclosed by the scanner, in particular the magnet unit. A field of view (FOV) and/or an isocenter of the magnetic resonance device is preferably located within the patient receiving region. The FOV preferably comprises a detection region of the magnetic resonance device within which the conditions for acquiring medical image data, in particular magnetic resonance image data, are present within the patient receiving region, such as, for example, a homogeneous main magnetic field. The isocenter of the magnetic resonance device preferably comprises the region and/or point within the magnetic resonance device that has the optimal and/or ideal conditions for acquiring medical image data, in particular magnetic resonance image data. In particular, the isocenter comprises the most homogeneous magnetic field region within the magnetic resonance device.

The magnetic resonance device further comprises a system controller, wherein the system controller comprises at least one computing module and/or processor. The system controller is designed to control the individual components of the magnetic resonance device for at least one magnetic resonance data acquisition and/or magnetic resonance data reconstruction. Said system controller generates corresponding control commands and/or control data for the individual components of the magnetic resonance device as a function of software executed by means of the processor and/or the computing module. The magnetic resonance device also has a memory in which software for operating the magnetic resonance device is stored.

Thus, the system controller is designed in particular to execute computer-readable instructions for controlling the individual components of the magnetic resonance device. In particular, the system controller comprises a memory, wherein computer-readable information is stored on the memory, wherein the system controller is designed to load the computer-readable information from the memory and to execute the computer-readable information for controlling the individual components of the magnetic resonance device. The components of the system controller can be predominantly in the form of software components. However, especially when particularly fast calculations are involved, some of these components can basically also be implemented in the form of software-supported hardware components, e.g. FPGAs or the like.

Of course, it is also conceivable for a plurality of the components mentioned to be implemented in a combined manner in the form of a single software component or software-supported hardware component.

An application addresses a clear clinical and/or medical objective, such as “I want to acquire diffusion images of the prostate and calculate an ADC map from them”. Different applications can therefore be available for different clinical and/or medical objectives, wherein the individual applications are independent of each other and each of the applications preferably addresses a single clinical and/or medical issue. Said individual applications are independent of each other, so that the individual applications can be individually tested and/or distributed and/or installed and/or executed and/or uninstalled. In addition, the individual applications can also be contained in application packages. Such an application package preferably comprises two or more applications, wherein within the application package a sequence and/or a workflow of the individual applications, in particular of an execution of the individual applications, is successively defined. For example, an application package may comprise an application involving a first magnetic resonance data acquisition and an application involving a reconstruction of the first magnetic resonance data, as well as an application involving a second magnetic resonance data acquisition and an application involving a reconstruction of the second magnetic resonance data, and a workflow application, wherein the workflow application controls the execution of the individual applications, in particular the order of the individual applications. Information and/or data is preferably exchanged between the individual applications of an application package.

For the purposes of the disclosure, the at least one application shall be understood as meaning in particular a single application or also a plurality of applications or also an application package comprising a plurality of applications.

The at least one application is preferably stored and/or provided in a separately implemented memory. In particular, the memory is implemented separately from the memory of the magnetic resonance device in which software of the magnetic resonance device is stored. Said memory in which the at least one application is stored and the memory for the software of the magnetic resonance device can, for example, be disposed on the same hard disk, the two regions and/or memory units being implemented independently and/or separately from one another. Exchange of data and/or exchange of information between the memory in which the at least one application is stored and the system controller of the magnetic resonance device is only possible by means of the interface. The at least one application can be stored and/or provided in a memory implemented as a cloud and/or as a network and/or as a server. The at least one application can also be available in a memory designed as a container for communication with the magnetic resonance device. Different applications can also be stored and/or provided in different storage locations and/or storage media, wherein each of the different applications can be connected to the magnetic resonance device by means of the interface in respect of exchanging information and/or exchanging data. The individual applications can also be installed and/or stored in a network-based manner such that, for example, the individual applications can be distributed by means of an application web store. The individual applications and/or application packages can comprise technical packages, such as a virtual machine and/or a docker container. The individual applications and/or application packages can also comprise a logical package. In particular, the interface and/or the magnetic resonance device provides a form of bookkeeping of the individual applications.

The at least one application is preferably formulated and/or designed generally such that the at least one application can be applied to different magnetic resonance devices. In particular, the at least one application is designed independently and/or non-specifically in terms of hardware properties and/or hardware information of magnetic resonance devices. Specification and/or matching to the magnetic resonance device in communication with the at least one application takes place only through the communication of the at least one application with the magnetic resonance device. By means of the communication between the at least one application and the magnetic resonance device, the information concerning the hardware properties of the magnetic resonance device is retrieved and/or obtained by the at least one application by means of the interface. By retrieving the information concerning the specific hardware properties, individual hardware-specific parameters of the at least one application can be specifically and/or individually matched to the magnetic resonance device.

The at least one application preferably comprises software that is designed and/or configured for execution on the magnetic resonance device. By means of the at least one application, a defined use of the magnetic resonance device can be controlled and/or executed. The at least one application preferably comprises, in a dedicated manner, at least the step of magnetic resonance data capture/acquisition and the step of magnetic resonance data reconstruction. The magnetic resonance data capture/acquisition preferably comprises the steps of scheduling magnetic resonance data acquisition and controlling magnetic resonance data acquisition, and is designed and/or configured to capture and/or acquire raw data. The magnetic resonance data acquisition of the at least one application can comprise at least one sequence and/or at least one protocol for a magnetic resonance examination.

In particular, a sequence for a magnetic resonance examination comprises a logical succession of radiofrequency (RF) pulses, gradient pulses, and acquisition periods for controlling data capture for magnetic resonance data acquisition. Said sequence specifies the main magnetic resonance mechanisms used in magnetic resonance data capture and or acquisition, such as, for example, a gradient echo or spin echo, a steady state of the magnetization, preparation pulses, etc. Typical and well-known sequences include a spin-echo sequence (SE sequence), a turbo spin-echo sequence (TSE sequence), an echo planar imaging (EPI) sequence, etc. However, a sequence does not define the complete time characteristics of all the RF pulses, gradient pulses and acquisition periods, but only their interaction. In principle, parameterization of a sequence can be adjusted. In particular, the timing of the sequence and/or resolution of the sequence can be adapted to the clinical and/or medical objective.

A protocol comprises a set of values that is required to concretize a sequence. Typical values can include an echo time (TE), a repetition time (TR), a field of view, a matrix size, a number of slices, a fat saturation method, an acceleration method, a local radiofrequency antenna, etc. Such protocol values are often also highly dependent on the locally available hardware and/or design of the magnetic resonance device, such as a gradient strength of the magnetic resonance device and/or a number of acquisition channels, etc. A protocol thus describes a very specific way of acquiring an image using a very specific embodiment of a magnetic resonance device. In particular, a contrast and/or a geometry and a measurement duration are matched to the clinical objective of the upcoming magnetic resonance examination.

The magnetic resonance data reconstruction of the at least one application is preferably designed and/or configured to reconstruct magnetic resonance image data and/or other results from the acquired raw magnetic resonance data. For this purpose, the at least one application can comprise an evaluation algorithm and/or a reconstruction algorithm that detects and/or determines magnetic resonance image data and/or other results from acquired raw magnetic resonance data. The raw magnetic resonance data is preferably acquired by means of the magnetic resonance data acquisition of the at least one application. Moreover, the at least one application can also comprise further applications, such as post-processing steps and/or workflow steps. A post-processing application may comprise, for example, an algorithm that further processes already reconstructed magnetic resonance image data. New image data and/or further results can be determined and/or obtained from the already reconstructed magnetic resonance image data. In addition, a workflow of an application and/or an application package may comprise a control, in particular a flow control and/or an information flow, between individual sequences and/or reconstructions.

The interface preferably comprises a universal interface by means of which applications can communicate and/or exchange data with a magnetic resonance device from different storage locations. Communication and/or data exchange between the at least one application and the magnetic resonance device is conducted exclusively by means of the interface. The interface is preferably designed to be scanner-independent, so that such an interface can be used for differently designed and/or structured magnetic resonance devices. In particular, the interface can be used for all scanner types in magnetic resonance devices, wherein the interface provides the corresponding hardware information for the applications. In particular, the interface has an interface element disposed on the magnetic resonance device and a corresponding interface element on the application side.

Communication and/or data exchange between the at least one application and the magnetic resonance device can take place both in the magnetic resonance device to the at least one application direction and in the at least one application to the magnetic resonance device direction. For this purpose, the interface preferably has a plurality of channels for information exchange and/or data exchange between the at least one application and the magnetic resonance device. These individual channels can comprise information channels by means of which information can be exchanged, and/or control channels by means of which control information, for example, control commands, can be exchanged, etc. This control information can also be processed into control commands only in the system controller of the magnetic resonance device. In particular, the interface represents an abstraction of the magnetic resonance device that provides all the relevant information, in particular hardware information, for the at least one application.

The at least one item of hardware information of the magnetic resonance device preferably comprises information about specific hardware properties of the magnetic resonance device, such as a hardware property of a main magnet, in particular a magnetic field strength of the main magnet, of the magnetic resonance device and/or a hardware property of a gradient system, in particular an available gradient strength of the gradient system, of the magnetic resonance device and/or a selection of local radiofrequency coils, etc. In particular, the at least one item of hardware information comprises a minimum hardware property required to adapt the at least one application for execution of the at least one application. Thus, the at least one item of hardware information can comprise a “low-level system description” of the magnetic resonance device that includes all the hardware information relevant to execution of the at least one application. By adjusting at least one parameter of the at least one application, the at least one application is individually matched to the magnetic resonance device, in particular to available hardware and/or current hardware limitations of the magnetic resonance device.

For execution of the at least one application on the magnetic resonance device, at least one item of control information, preferably a plurality of items of control information, are provided for the magnetic resonance device, in particular the system controller of the magnetic resonance device, by means of the interface, in particular by means of a control channel of the interface. Execution of the at least one application on the magnetic resonance device is controlled/monitored by the application, while the magnetic resonance device only provides the executing hardware for executing the at least one application. The at least one item of control information can comprise control information for controlling the gradient system, e.g. for playout of a gradient pulse, and/or control information for the radiofrequency unit, e.g. for playout/emission of an excitation pulse, and/or control information for detecting magnetic resonance signals, etc.

The disclosure can provide simple use, in particular simple installation and/or execution, of individual software components and/or applications. The installation and/or execution is independent of the installation status of the existing software of the magnetic resonance device.

In particular, the individual software components and/or applications can be called up individually for a specific clinical and/or medical objective. Because of the independence of the individual applications and/or individual application packages, an operator of a magnetic resonance device, for example, can also merely purchase individual applications and/or individual application packages without having to reinstall the complete software. In addition, because of the independence of the individual applications and/or the individual application packages, a plurality of versions of an application can also be available for the magnetic resonance device, a selection mechanism preferably being available for this purpose.

In addition, the design of the applications also allows them to be used with different magnetic resonance devices, so that additional development costs can advantageously be saved. Moreover, applications from other suppliers can also be easily and quickly implemented on the magnetic resonance device.

Another advantage of the disclosure is that if a new hardware functionality is developed, the interface can be provided with a new function and the “old version” of the interface can continue to be available, thereby obviating the need to completely replace the applications.

In an advantageous development of the method according to the disclosure, it can be provided that the interface is designed to provide the information and/or data required for the at least one application and to provide the information and/or data required to execute the at least one application to the magnetic resonance device. The interface is preferably designed to be scanner-independent, so that the interface can be used in magnetic resonance devices having different designs, in particular hardware designs, such as, for example, magnetic field strength and/or a maximum gradient strength and/or radiofrequency system design, etc. Moreover, the interface is also application-independent, so that the interface can be used for applications having different embodiments and/or designs. In particular, information can be exchanged between the magnetic resonance device and the at least one application only by means of the interface.

The interface constitutes in particular an abstraction of the magnetic resonance device that provides all the relevant information, in particular hardware information, for the at least one application. The interface comprises a type of portal and/or platform for communication, in particular information exchange and/or data exchange, between the at least one application and the magnetic resonance device, in particular the system controller of the magnetic resonance device.

The interface is designed to provide the at least one application with all the relevant information, in particular the hardware design of the magnetic resonance device. For this purpose, the interface can have a memory in which the relevant information of the magnetic resonance device is stored and/or deposited. For example, the relevant information is stored in a table and/or a database. It is also possible for the table and/or database to be dynamically adapted, as may be the case e.g. in the event of changing hardware information. In particular, the field homogeneity inside the patient receiving region may be subject to change as a result of the patient being moved into the patient receiving region. The field inhomogeneity may also be different for different patients within the patient receiving region of the magnetic resonance device. The interface is preferably designed to provide the at least one application with all the relevant and/or necessary information and/or data required for executing the at least one application on the magnetic resonance device.

In addition, all the information and/or data required for executing the at least one application on the magnetic resonance device is provided to the magnetic resonance device, in particular the system controller of the magnetic resonance device, by means of the interface. In particular, control information for controlling the gradient system and/or the radiofrequency system can be provided to the system controller in this manner.

In particular, the interface is designed to be constant over time so that communication between the at least one application and a magnetic resonance device can take place regardless of the age of the at least one application and/or regardless of the age of the magnetic resonance device. Moreover, for a new functionality of the magnetic resonance device, it is possible for a new interface upgraded with this functionality to be provided. However, even applications that do not run this new functionality can still communicate with the magnetic resonance device via this new interface.

For communication purposes, the interface preferably has a plurality of channels, such as e.g. at least one information channel and/or at least one control channel and/or other channels for data exchange and/or information exchange that will be expedient to persons skilled in the art.

In an advantageous development of the method according to the disclosure, it can be provided that the at least one item of hardware information relating to the magnetic resonance device comprises magnet information relating to a main magnet and/or gradient information relating to a gradient system and/or transmit information relating to a radiofrequency system and/or receive information relating to the radiofrequency system and/or information relating to a reconstruction unit (reconstructor). The at least one item of hardware information preferably comprises a minimum hardware property, in particular a “low-level system description” of the magnetic resonance device, which is required for adapting the at least one application for execution of the at least one application.

The magnet information relating to the main magnet can include a magnetic field strength, for example. The gradient information relating to the gradient system can include a maximum available gradient field strength, for example. Said maximum available gradient field strength may be limited by the hardware, i.e. by the gradient coils of the gradient system, or may be limited by restrictions and/or licenses that specify a maximum gradient strength.

The radiofrequency system preferably comprises the radiofrequency antenna and also the local radiofrequency coils disposed locally on the region of the patient to be examined. The transmit information of the radiofrequency system can include the type and/or power of the radiofrequency antenna, for example. The receive information of the radiofrequency system can include, for example, the number of local radiofrequency coils connected to the magnetic resonance device and/or the type of local radiofrequency coils connected to the magnetic resonance device and/or the coil selection of the local radiofrequency coils connected to the magnetic resonance device and/or information relating to the receive electronics, etc.

The reconstructor information can include, for example, system information relating to a reconstruction computer.

In this way, the at least one application can advantageously be individually matched to the hardware of the magnetic resonance device available on site. In particular, a universally available application can be quickly and easily matched to the individual design of a magnetic resonance device and thus the uses available to an operator and/or user of the magnetic resonance device can be advantageously increased.

In an advantageous development of the method according to the disclosure, it can be provided that, by means of at least one information channel of the interface, information provided by the at least one application is made available for display on a user interface of the magnetic resonance device. The information provided can be transmitted by the interface directly to the user interface or forwarded to the user interface via the system controller.

The information provided can include, for example, information about a pending data acquisition and/or a current execution status of the at least one application. In addition, the at least one item of information can also include one or more prompts for the user, in particular for medical staff. For example, settable parameters can be adjusted by the user to suit the clinical objective and/or a start input can be entered to launch the application, etc.

The user interface of the magnetic resonance device preferably comprises an information presentation and/or display comprising e.g. a monitor and/or display. In addition, the user interface also comprises an input by means of which an input, in particular a user input, can be made. The input can comprise a keyboard and/or a computer mouse.

It is also possible for the display and the input of the user interface to be embodied together at least partially as one part and/or one piece, e.g. by means of a touch display and/or a touch-sensitive sensor screen.

This embodiment of the disclosure enables advantageous transmission of information to the user to take place. In particular, the user can be informed in this way, for example, about individual segments during execution of the at least one application.

In an advantageous development of the method according to the disclosure, it can be provided that information concerning a user interaction in respect of the information relating to the at least one application provided by the at least one application is made available by means of the at least one information channel of the interface. It can be provided that the information provided by the at least one application provides for and/or permits a defined user interaction with the user interface. The user interaction preferably comprises a user input. The information relating to a user interaction preferably comprises a value and/or a result of the user interaction and/or of the user input at the user interface. Said user interaction can comprise an input made to the user interface on the basis of a prompt contained in the information provided. For example, a user can inform the system that all the preparations for the upcoming execution of the at least one application have been completed and the at least one application can now be executed. In addition, the user can be specifically prompted to enter a user interaction with the at least one application by means of the user interface.

In an advantageous development of the method according to the disclosure, it can be provided that the information provided by the at least one application comprises a graphical interface of a display, wherein the graphical interface can be displayed by means of the user interface of the magnetic resonance device. Here the magnetic resonance device provides the executing hardware, e.g. a monitor and/or a display of the user interface of the magnetic resonance device. The display, in particular the display of the graphical user interface, is controlled by means of the at least one application. In particular, all the user inputs required for execution of the at least one application can be requested particularly simply and directly by the user in this way. In addition, the user can also be informed directly and quickly about the execution of the at least one application in this way.

In an advantageous development of the method according to the disclosure, it can be provided that the at least one application is scanner-independent. A scanner-independent application is to be understood as meaning in particular that the application is designed independently of the technical properties of a scanner of a magnetic resonance device. Specification and/or an adaptation to the magnetic resonance device in communication with the at least one application takes place only by providing the at least one item of hardware information for the at least one application. This means that the at least one application can be provided for use on different magnetic resonance devices and/or scanners of magnetic resonance devices, thereby enabling scanner-independent use of the at least one application. Furthermore, the at least one application can always be adapted particularly quickly and easily to suit current hardware limitations of the magnetic resonance device.

In an advantageous development of the method according to the disclosure, it can be provided that, for execution of the at least one application, the at least one application provides control information for controlling at least one hardware component of the magnetic resonance device by means of the control channel of the interface. The at least one hardware component of the magnetic resonance device comprises, for example, the gradient system and/or a radiofrequency receive amplifier and/or a patient table of the magnetic resonance device. Said control information may already comprise control commands that can be directly executed by the system controller of the magnetic resonance device for controlling the at least one hardware component. In addition, the control information can also first be converted within the system controller into a control command for controlling the at least one hardware component. In particular, the at least one application preferably provides control information for all the hardware components required for execution of the at least one application, for example, for acquiring magnetic resonance data, by means of the control channel of the interface. This allows the individual hardware components of the scanner of the magnetic resonance device to be optimally controlled during execution of the at least one application.

In an advantageous development of the method according to the disclosure, it can be provided that, prior to execution of the at least one application and/or during execution of the at least one application, dynamic control information concerning the magnetic resonance device is made available to the at least one application by means of an information channel of the interface. Dynamic control information is to be understood in particular as meaning information that is acquired dynamically before and/or during execution of the at least one application and that is taken into account by the at least one application for controlling execution of the at least one application. For example, the dynamic control information can comprise a patient's ECG data which is acquired from the patient during execution of the at least one application. Such ECG data can be used in particular as a trigger signal for data acquisition during execution of the at least one application. The dynamic control information may also comprise a respiratory signal of the patient and/or movement information of the patient that is detected during execution of the at least one application, and/or other dynamically detectable information that will be deemed useful by persons skilled in the art. In this way, the execution of the at least one application can be advantageously adapted to a patient's state and/or to dynamically changing conditions and/or situations during execution of the at least one application.

In an advantageous development of the method according to the disclosure, it can be provided that raw data of a magnetic resonance measurement is provided by means of an information channel of the interface for reconstruction of the at least one application. The raw data preferably comprises raw magnetic resonance data acquired by means of the magnetic resonance device. The raw data can also comprise raw data from tuning measurements, such as measurements relating to coil sensitivities of local radiofrequency coils, for example. The raw data has preferably been previously acquired by using the at least one application on the magnetic resonance device. The reconstruction of the at least one application is preferably geared to the clinical and/or medical objective. In particular, an evaluation algorithm and/or a reconstruction algorithm of the at least one application is geared to the clinical and/or medical objective, so that the reconstruction of the raw data by means of the at least one application allows particularly efficient and fast reconstruction of the raw data in respect of the clinical and/or medical objective.

In an advantageous development of the method according to the disclosure, it can be provided that reconstructed magnetic resonance image data is provided by the at least one application by means of the information channel of the interface. The reconstructed magnetic resonance image data is preferably provided immediately after reconstruction of the raw data by means of the at least one application. In this way, a user can obtain magnetic resonance image data particularly quickly for further use. In particular, a user can obtain magnetic resonance image data in this manner, wherein the reconstruction of the magnetic resonance data is specifically geared to the clinical and/or medical objective, thereby facilitating diagnosis and/or further processing for the user.

In an advantageous development of the method according to the disclosure, it can be provided that the interface is provided directly on a system controller of the magnetic resonance device and/or is connected to the system controller of the magnetic resonance device by means of a network. The network preferably comprises a data network for the exchange of data. Said network can be comprised by the magnetic resonance device. In addition, it is also conceivable for the network to be a local network, such as a hospital network, for example. In this way, the interface can be integrated particularly easily into a locally available infrastructure for a magnetic resonance device. In addition, the system controller can exchange data directly with the interface in this way.

In an advantageous development of the method according to the disclosure, it can be provided that the interface has a license module. In this context, a license module is to be understood in particular as meaning a module of the interface that preferably manages licenses and/or access rights held by the magnetic resonance device in respect of specific uses and/or applications. In addition, access rights of the individual applications for controlling individual parts of the magnetic resonance device can also be managed by means of the license module. The license module of the interface allows differentiation between individual applications by managing information exchange and/or data exchange of information and/or data provided by means of the interface between the magnetic resonance device and separate applications individually, in particular on an application-dependent basis. For example, when a hardware property of the gradient system is communicated, a diffusion application can receive information with a maximum gradient strength of the gradient system and a perfusion application can receive only information with a reduced gradient strength of the gradient system. In addition, extension of a license, by acquisition of additional rights, for example, can be easily managed in the license module.

In addition, the license module can also be designed to detect application usage by a magnetic resonance device, in particular by a user and/or operator of a magnetic resonance device. For example, a license can be provided with a limitation on uses of an application and/or with a limitation on hardware performance of a hardware component of the magnetic resonance device, such as the gradient system of the magnetic resonance device, for example. In addition, a limitation on use of an application can also be designed such that a user and/or an operator of a magnetic resonance device can use a low level of the application without additional costs, whereas higher level usage of the application is recorded individually and must also be paid for in each case. For example, a low level of an application can involve only low capacity utilization of the hardware components of the magnetic resonance device, such as the gradient system, for example, while the higher levels of the application can involve correspondingly higher capacity utilization of the hardware components of the magnetic resonance device and/or further additional services.

In an advantageous development of the method according to the disclosure, it can be provided that the interface has a learning module, wherein the learning module comprises an artificial neural network. The learning module, in particular the artificial neural network, is preferably designed to adapt the at least one application to the magnetic resonance device, in particular to hardware properties and/or to license limitations of the magnetic resonance device.

The adaptation of the at least one application to the magnetic resonance device, in particular to hardware properties and/or to license limitations of the magnetic resonance device, is preferably based on a machine learning method, also termed a deep learning method, that is based on the artificial neural network. An artificial neural network (ANN) is in particular a network of artificial neurons simulated in a computer program. The artificial neural network is typically based on an interconnection of a plurality of artificial neurons. The artificial neurons are typically disposed on different layers. The artificial neural network usually comprises an input layer and an output layer whose neuron output is the only one visible in the artificial neural network. Layers between the input layer and the output layer are typically referred to as hidden layers. Typically, an artificial neural network architecture and/or topology is first initiated and then trained for a specific task or for multiple tasks in a training phase. The training of the artificial neural network typically involves changing the weight of a connection between two artificial neurons of the artificial neural network. The training of the artificial neural network may also include developing new connections between artificial neurons, removing existing connections between artificial neurons, adjusting thresholds of the artificial neurons, and/or adding or removing artificial neurons.

In particular, the artificial neural network will already have been suitably trained in advance to adapt the at least one application to the magnetic resonance device, in particular to hardware properties and/or to license limitations of the magnetic resonance device. In particular, for training the artificial neural network, training data sets will have been used, e.g. training data which places a minimum requirement on the hardware properties of the magnetic resonance device.

In this way, fast individual and/or specific adaptations of the at least one application, in particular of protocols and/or sequences of the at least one application, can be advantageously achieved. A further advantage is that the individual applications do not have to be customized for any specific hardware property of magnetic resonance devices during their development, but that the applications can be kept general and/or universal. This significantly reduces the development effort and thus also the development costs for the individual applications.

In addition, the disclosure proceeds from a magnetic resonance device having an interface, wherein, by means of the interface, the magnetic resonance device is designed to carry out the method for executing at least one application on a magnetic resonance device.

The disclosure can provide ease of use, in particular simple installation and/or execution, of individual software components and/or applications. In particular, the individual software components and/or applications can be called up individually for a specific clinical and/or medical objective. Also, due to the independence of the individual applications and/or individual application packages, an operator of a magnetic resonance device can, for example, purchase only individual applications and/or individual application packages without having to reinstall the complete software.

In addition, the design of the applications also allows them to be used with different magnetic resonance devices, so that additional development costs can be saved. Moreover, applications from other suppliers can also be easily and quickly implemented on the magnetic resonance device.

The advantages of the magnetic resonance device according to the disclosure essentially correspond to the advantages of the method according to the disclosure for executing at least one application on a magnetic resonance device, as detailed above. Features, advantages or alternative embodiments mentioned here are likewise applicable to the other described subject matters and vice versa.

Furthermore, the disclosure is based on a system with a magnetic resonance device and at least one application, wherein the system is designed to carry out the method for executing at least one application on a magnetic resonance device.

The advantages of the system according to the disclosure essentially correspond to the advantages of the method according to the disclosure for executing at least one application on a magnetic resonance device, as detailed above. Features, advantages or alternative embodiments mentioned here are likewise applicable to the other described subject matter and vice versa.

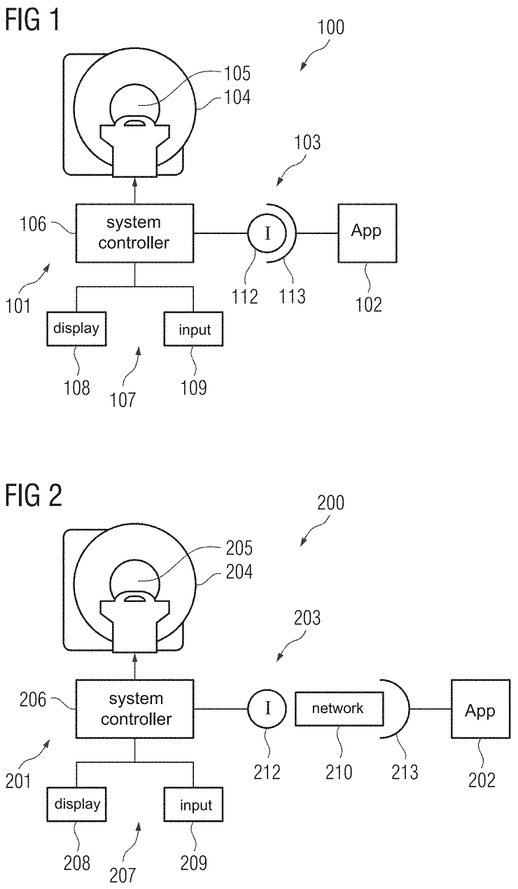

FIGS. 1 to 3 respectively show a system 100, 200, 300 with a magnetic resonance device 101, 201, 301 and an application 102, 202, 302, wherein the magnetic resonance device 101, 201, 301 communicates and/or exchanges data and/or information with the application 102, 202, 302 by means of an interface 103, 203, 303.

The magnetic resonance device 101, 201, 301 comprises in each case a scanner 104, 204, 304 and a patient receiving region 105, 205, 305 cylindrically enclosed by the scanner 104, 204, 304 for accommodating a patient. The scanner 104, 204, 304 in FIGS. 1 to 3 is shown only schematically and comprises a superconducting main magnet, a gradient system, and a radiofrequency system as known from the prior art. The main magnet is designed to generate a powerful and in particular constant main magnetic field. The gradient system is designed to generate magnetic field gradients that are used for spatial encoding during imaging. The radiofrequency system has a radiofrequency antenna designed to excite a polarization that arises in the main magnetic field generated by the main magnet.

To control the main magnet, the gradient system, and the radiofrequency antenna, the magnetic resonance device 101, 201, 301 comprises a respective system controller 106, 206, 306. The system controller 106, 206, 306 is configured to centrally control the magnetic resonance device 101, 201, 301. In an exemplary embodiment, the system controller 106, 206, 306 includes processing circuitry that is configured to perform one or more functions and/or operations of the system controller 106, 206, 306, including controlling the main magnet, the gradient system, and the radiofrequency antenna, processing magnetic resonance signals, reconstructing magnetic resonance images, processing input from the user of the magnetic resonance imaging device, providing an output to the user, and/or controlling the overall operation of the magnetic resonance device 101, 201, 301.

The magnetic resonance device 101, 201, 301 also comprises a user interface 107, 207, 307 which is connected to the system controller 106, 206, 306. Control information, such as imaging parameters, as well as reconstructed magnetic resonance images can be displayed on a display 108, 208, 308, for example, on at least one monitor, of the user interface 107, 207, 307 for medical staff. In addition, the user interface 107, 207, 307 comprises an input 109, 209, 309 by means of which information and/or parameters can be entered by medical staff during a measurement process.

The interface 103, 203, 303 of the system 100, 200, 300 is designed here as a universal interface 103, 203, 303, so that different applications 102, 202, 302 and/or software packages can communicate and/or exchange data with the magnetic resonance device 101, 201, 301 by means of the interface 103, 203, 303. In particular, said interface 103, 203, 303 is designed to be both scanner-independent and application-independent. The interface 103, 203, 303 comprises a type of platform or portal for communication, in particular information exchange and/or data exchange, between the application 102, 202, 302 and the magnetic resonance device 101, 201, 301, in particular the system controller 106, 206, 306 of the magnetic resonance device 101, 201, 301. For this purpose, the interface 103, 203, 303 has a first interface element 112, 212, 312 which is disposed on the magnetic resonance device and a second, corresponding interface element 113, 213, 313 which is disposed on the application side.

The information exchange and/or the data exchange between the application 102, 202, 302 and the magnetic resonance device 101, 201, 301 can take place both in the direction from the magnetic resonance device 101, 201, 301 to the at least one application 102, 202, 302 and in the direction from the at least one application 102, 202, 302 to the magnetic resonance device 101, 201, 301 by means of the interface 103, 203, 303. In particular, the information exchange and/or the data exchange by means of the interface 103, 203, 303 can be bidirectional. For this purpose, the interface 103, 203, 303 preferably has a plurality of channels for information exchange and/or data exchange between the application 102, 202, 302 and the magnetic resonance device 101, 201, 301. These individual channels can comprise information channels, by means of which information can be exchanged, and/or control channels, by means of which control information, e.g. control commands, can be exchanged, etc.

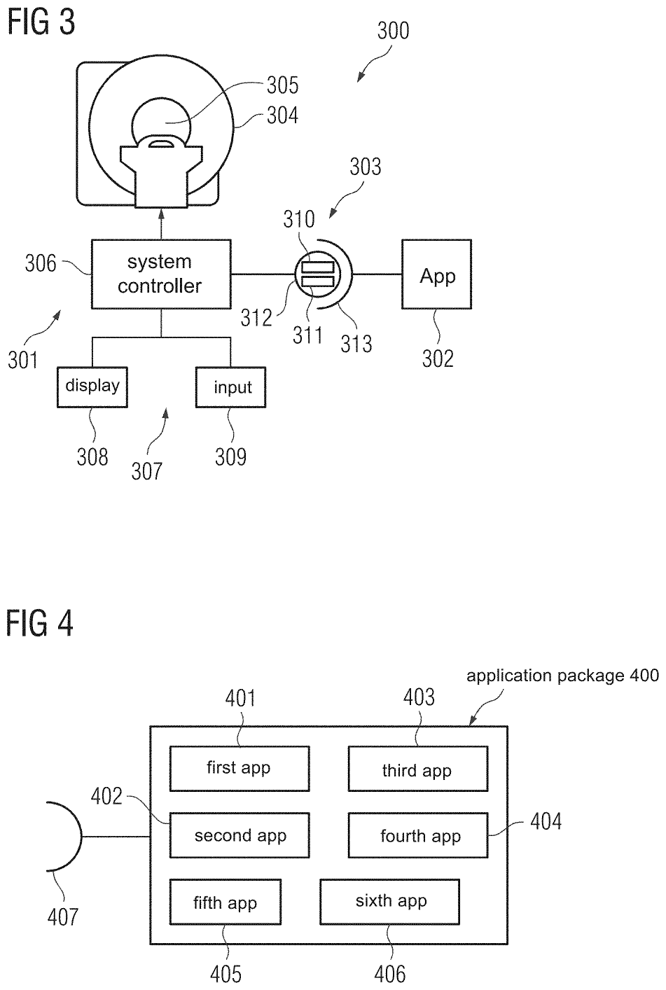

The systems 100, 200, 300 shown in FIGS. 1 to 3 differ in respect of the design of the interface 103, 203, 303. In contrast, the magnetic resonance devices 101, 201, 301 and the applications 102, 202, 302 are of essentially identical structure and/or design. In this regard, the interfaces 103, 303 in FIGS. 1 and 3 are linked directly to the system controller 106, 306 of the magnetic resonance device 101, 301. The interface 203 in FIG. 2 is connected to the magnetic resonance device 201, in particular the system controller 206 of the magnetic resonance device 201, by means of a network 210. In FIG. 3, the interface 303 also has a license module 310 and a learning module 311. The license module 310 and the learning module 310 are incorporated in the interface element 312 of the magnetic resonance device 301. The mode of operation of these two modules will be described in further detail below.

The application 102, 202, 302 is stored and/or deposited on a separately implemented memory. The at least one memory is preferably implemented separately from a system controller 106, 206, 306 and separately from a memory of the magnetic resonance device 101, 201, 301 in which software of the magnetic resonance device 101, 201, 301 is stored. The memory in which the application 102, 202, 302 is stored and/or deposited can also be comprised by a cloud and/or a network and/or a server and/or a container.

An application 102, 202, 302 is geared to a clear clinical and/or medical objective. Thus, different applications 102, 202, 302 may also be available for different clinical and/or medical objectives, wherein the individual applications 102, 202, 302 are independent of one another and each of the applications 102, 202, 302 addresses its own clinical and/or medical objective.

In FIG. 4, an application package 400 is shown in more detail. The application package 400 preferably comprises software designed and/or configured for execution on the magnetic resonance device 101, 201, 301. The application package in FIG. 4 comprises a plurality of individual applications 401, 402, 403, 404, 405, 406 and an interface element 407. By means of the respective application 401, 402, 403, 404, 405, 406, a defined application of the magnetic resonance device 101, 201, 301 can be controlled and/or executed. The application package 400 comprises two applications 401, 402 for magnetic resonance data capture and/or magnetic resonance data acquisition and two applications 403, 404 for magnetic resonance data reconstruction 403, 404. The two applications 401, 402 for magnetic resonance data acquisition preferably comprise the steps of scheduling data acquisition on the magnetic resonance device 101, 201, 301 and controlling data acquisition on the magnetic resonance device 101, 201, 301 for capture and/or acquisition of raw data. The two applications 401, 402 of magnetic resonance data acquisition comprise at least one sequence and/or at least one protocol for a magnetic resonance examination.

The two applications 403, 404 for magnetic resonance data reconstruction preferably comprise an evaluation algorithm and/or a reconstruction algorithm that obtains magnetic resonance image data and/or further results from the acquired raw magnetic resonance data. Another application 405 of the application package 400 comprises a post-processing application. Such a post-processing application can comprise an algorithm, in particular a post-processing algorithm, which generates and/or determines new image data and/or further results from magnetic resonance image data. The application package 400 additionally comprises a further application 406, wherein the further application 406 comprises a workflow application. The workflow application is preferably designed to control a flow and/or an execution sequence of the individual applications 401, 402, 403, 404, 405 of the application package and/or to control an exchange of information between the individual applications 401, 402, 403, 404, 405. Moreover, in an alternative embodiment, the application package 400 can also comprise further applications that will be deemed useful by persons skilled in the art.

The individual applications 401, 402, 403, 404, 405, 406 are designed to be scanner-independent. In particular, the individual applications 401, 402, 403, 404, 405, 406 are designed/structured independently of individual properties, such as hardware properties, for example, and/or the technical design of magnetic resonance devices 101, 201, 301. Individual and/or specific adaptation of the individual application 401, 402, 403, 404, 405, 406, in particular individual and/or specific adaptation of the sequences and/or protocols of the individual applications 401, 402, 403, 404, 405, 406, takes place only through an exchange of data and/or an exchange of information with the magnetic resonance device 101, 201, 301 by means of the interface 103, 203, 303.

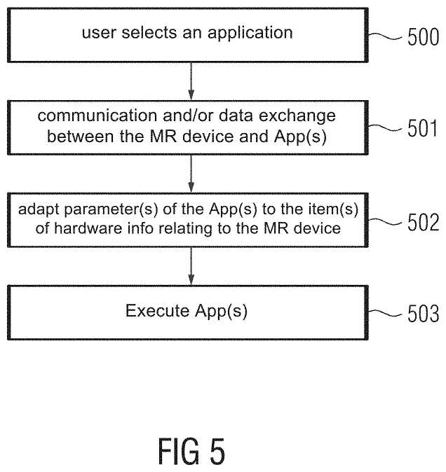

FIG. 5 shows a method according to the disclosure for executing an application 102, 202, 302 on a magnetic resonance device 101, 201, 301. There is a specific medical and/or clinical objective for a magnetic resonance examination on a patient. This medical and/or clinical question involved is to be clarified by means of acquired magnetic resonance data which is acquired by means of the magnetic resonance examination on the patient, in particular on the region of the patient to be examined. For this purpose, medical staff, for example a physician supervising the magnetic resonance examination, have one or more examination procedures at their disposal. In the present case, in a first step 500, the user selects an application 102, 202, 302 which is stored on a separately implemented memory as described above. This application 102, 202, 302 can be executed by means of the user interface 107, 207, 307 of the magnetic resonance device 101, 201, 301. The selection is controlled by the system controller 106, 206, 306 of the magnetic resonance device 101, 201, 301.

As soon as an application 102, 202, 302 or also a plurality of applications 102, 202, 302 or an application package 400 has been selected by the user, in a further step 501, communication and/or data exchange takes place between the magnetic resonance device 101, 201, 301 and the at least one application 102, 202, 302, in particular the one application 102, 202, 302 or also the plurality of applications 102, 202, 302. At least one item of hardware information relating to the magnetic resonance device 101, 201, 301 is provided to the at least one application 102, 202, 302 by means of an information channel of the interface 103, 203, 303. The at least one item of hardware information is provided automatically by means of the at least one application 102, 202, 302 and/or by means of the interface 103, 203, 303. For providing the at least one item of hardware information, the interface 103, 203, 303 can also comprise a database and a table and/or access a database and/or a table stored in a memory of the interface 103, 203, 303 and/or of the system controller 106, 206, 306. Here, the interface 103, 203, 303 constitutes an abstraction of the magnetic resonance device 101, 201, 301 that provides all the relevant information, in particular hardware information, for the at least one application 103, 203, 303.

The at least one item of hardware information of the magnetic resonance device 101, 201, 301 comprises specific and/or individual hardware information relating to the magnetic resonance device 101, 201, 301. The at least one item of hardware information relating to the magnetic resonance device 101, 201, 301 can include magnet information relating to the main magnet of the magnetic resonance device 101, 201, 301. The magnet information preferably comprises the magnetic field strength of a main magnetic field generated and/or produced by means of the main magnet. For example, the magnetic field strength can be 1.5 T or 3 T, etc.

In addition, the at least one item of hardware information can also comprise gradient information relating to the gradient system. The gradient information relating to the gradient system can comprise, for example, a maximum available gradient field strength. Said maximum available gradient field strength may be predefined by the hardware, i.e. by gradient coils of the gradient system, or may also be limited by restrictions and/or licenses that specify a maximum gradient field strength that can be played out.

In addition, the at least one item of hardware information can also comprise transmit information relating to the radiofrequency system and/or receive information relating to the radiofrequency system. The radiofrequency system preferably comprises the radiofrequency antenna and also local radiofrequency coils disposed locally on the region of the patient under examination. The transmit information relating to the radiofrequency system can comprise, for example, the type and/or power of the radiofrequency antenna. The receive information relating to the radiofrequency system can include, for example, the number of local radiofrequency coils connected to the magnetic resonance device and/or the type of local radiofrequency coils connected to the magnetic resonance device and/or coil selection of the local radiofrequency coils connected to the magnetic resonance device and/or information relating to receive electronics, etc.

The hardware information relating to the magnetic resonance device 101, 201, 301 can also include reconstructor information. For example, the reconstructor information can include system information relating to a reconstruction computer.

Then, in a subsequent step 502, at least one parameter of the at least one application 102, 202, 302 is adapted to the at least one item of hardware information relating to the magnetic resonance device 101, 201, 301. By adjusting the at least one parameter, the at least one application 102, 202, 302 is individually adapted to the hardware and/or magnetic resonance device 101, 201, 301 available locally for executing the at least one application 102, 202, 302 on the magnetic resonance device 101, 201, 301.

Then, in a further step 503, the at least one application 102, 202, 302 is executed, wherein, by means of a control channel of the interface 103, 203, 303, at least one item of control information is provided by the at least one application 102, 202, 302 for the magnetic resonance device 101, 201, 301 for executing the at least one application 102, 202, 302. Execution of the at least one application 102, 202, 302 preferably involves detecting or acquiring magnetic resonance data and reconstructing the acquired magnetic resonance data. In addition, execution of the at least one application 102, 202, 302 can also involve post-processing steps or workflow steps, etc. The at least one item of control information provided by the at least one application 102, 202, 302 via the interface 103, 203, 303 is sent to the system controller 106, 206, 306 which locally controls the individual components of the magnetic resonance device 101, 201, 301 according to the instructions provided by the at least one application 102, 202, 302. For example, when the at least one application 102, 202, 302 is executed, direct activation of the gradient system of the magnetic resonance device 101, 201, 301 and/or the radiofrequency system and/or positioning of a patient table of the magnetic resonance device 101, 201, 301 is controlled by the system controller 106, 206, 306 by means of the control information provided. Control information is preferably provided by the at least one application 102, 202, 302 to the system controller 106, 206, 306 via the control channel of the interface 103, 203, 303 for all hardware components required for executing the at least one application 102, 202, 302.

During execution of the at least one application 102, 202, 302, information provided by the at least one application 102, 202, 302 can also be provided for display on the user interface 107, 207, 307 of the magnetic resonance device 101, 201, 301 by means of the information channel of the interface 103, 203, 303. In particular, the at least one application 102, 202, 302 determines which parameters and/or which items of information are displayed or presented to the user, in particular medical operating staff, on the display 108, 208, 308 of the user interface 107, 207, 307. Only parameters and/or information displayed on a graphical user interface provided by the user interface 107, 207, 307 and/or the system controller 106, 206, 306 of the magnetic resonance device 101, 201, 301 by means of the display 108, 208, 308 can be selected by the at least one application 102, 202, 302. In addition, it can also be the case that the information provided by the at least one application 102, 202, 302 also comprises a graphical interface of a display provided by the at least one application 102, 202, 302 for display and/or presentation on the user interface 107, 207, 307 of the magnetic resonance device 101, 201, 301.

In addition, a user interaction in respect of the information provided by the at least one application 102, 202, 302 can be transmitted and/or provided to the at least one application 102, 202, 302 by means of the at least one information channel of the interface 103, 203, 303. Here it can be provided that the information provided by the at least one application 102, 202, 302 allows and/or requires a defined user interaction with the user interface. For example, the information provided by the at least one application can also comprise a prompt to the user, said prompt resulting in a user interaction, in particular an input, at the user interface 107, 207, 307 of the magnetic resonance device 101, 201, 301. For example, a user can inform the at least one application 102, 202, 302 that all the preparations for the upcoming execution of the at least one application 102, 202, 302 have been completed and that the at least one application 102, 202, 302 can now be executed. A user interaction can also include a start input for starting magnetic resonance data acquisition. Such a start input can, for example, be dependent on the patient's position and/or an arrangement of accessory units that only the user needs to check locally. In addition, in this way the user can also be specifically prompted to enter a user interaction with the at least one application 102, 202, 302 by means of the user interface 107, 207, 307.

In addition, by means of the information channel of the interface 103, 203, 303, dynamic control information can be provided by the magnetic resonance device 101, 201, 301 to the at least one application 102, 202, 302 prior to execution of the at least one application 102, 202, 302 and/or also during execution of the at least one application 102, 202, 302. Such dynamic control information comprises information that is preferably continuously acquired and which is taken into account by the at least one application 102, 202, 302 for controlling execution of the at least one application 102, 202, 302. For example, the dynamic control information comprises ECG data of the patient and/or a respiratory signal of the patient and/or a heart rate of the patient and/or other continuously changing and/or dynamically changing information. Dynamic control information of this kind, such as the patient's ECG data, can be used as a trigger signal from the at least one application 102, 202, 302 for capture and/or acquisition of magnetic resonance data.

For data reconstruction, in particular magnetic resonance data reconstruction, raw data of a magnetic resonance measurement, in particular of capture and/or acquisition of magnetic resonance data, is provided by the magnetic resonance device 101, 201, 301 to the at least one application 102, 202, 302 by means of the information channel of the interface 103, 203, 303 when the at least one application 102, 202, 302 is executed. Within the at least one application 102, 202, 302, the raw data, in particular the raw magnetic resonance data, is then preferably reconstructed. In addition, the reconstructed magnetic resonance data, in particular magnetic resonance image data, can then be provided and/or transmitted by the at least one application 102, 202, 302 of the magnetic resonance device 101, 201, 301 by means of the information channel of the interface 103, 203, 303.

If the interface 303 also has a license module 310, as shown in FIG. 3, licenses and/or access rights can be managed by means of the interface 303, in particular by means of the license module 310 of the interface 303. The license module 310 of the interface 303 is used to manage licenses and/or access rights held by the magnetic resonance device 301 in respect of particular uses and/or applications 302, as well as access rights of the individual applications 302 for controlling individual parts of the magnetic resonance device 301. The license module 310 enables individual applications 302 to be differentiated within the interface 303 in that an exchange of information and/or an exchange of data between the magnetic resonance device 301 and individual applications 302 is managed individually, in particular in an application-dependent manner, by the license module 310. For example, when a hardware property of the gradient system is communicated, a diffusion application can receive information with a maximum gradient strength of the gradient system and a perfusion application can receive only information with a reduced gradient strength of the gradient system. In addition, an extension of a license, by purchasing of additional rights, for example, can be managed accordingly in the license module 310. The license module 310 can also be designed to record usage of applications 302, in particular the number of executions of an application 302.

If the interface 303 also has a learning module 311, as shown in FIG. 3, adaptation of the at least one application 302 to the magnetic resonance device 301, in particular to hardware properties and/or to license limitations of the magnetic resonance device 301, can be carried out autonomously and/or automatically by means of the interface 303, in particular the learning module 311 of the interface 303. The learning module preferably comprises a trained artificial neural network that is trained to adapt an application 302 to the hardware properties of the magnetic resonance device 301.

Although the disclosure has been illustrated and described in detail by the preferred embodiment, the disclosure is not limited by the examples disclosed and other variations will be apparent to persons skilled in the art without departing from the scope of protection sought for the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein. In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for executing at least one application on a magnetic resonance (MR) device, the method comprising:
storing the at least one application on a separately implemented memory;
performing a communication and/or data exchange with the at least one application using an interface of the MR device;
communicating at least one item of hardware information relating to the MR device to the at least one application using an information channel of the interface;
matching at least one parameter of the at least one application to the at least one item of hardware information of the MR device; and
providing at least one item of control information using a control channel of the interface, by the at least one application of the magnetic resonance device, to execute the at least one application.

2. The method as claimed in claim 1, wherein the interface is configured to:
provide information and/or data required for the at least one application; and
the information and/or data required to execute the at least one application to the MR device.

3. The method as claimed in claim 1, wherein the at least one item of hardware information comprises:
magnet information relating to a main magnet;
gradient information relating to a gradient system;
transmit information relating to a radiofrequency system;
receive information relating to the radiofrequency system; and/or information relating to a reconstruction processor.

4. The method as claimed in claim 1, wherein information provided by the at least one application is provided to a user interface of the magnetic resonance device using the at least one information channel of the interface.

5. The method as claimed in claim 4, wherein information relating to a user interaction associated with the information provided by the at least one application is supplied to the at least one application using the at least one information channel of the interface.

6. The method as claimed in claim 4, wherein the information provided by the at least one application comprises a graphical interface of a display, the graphical interface being displayable by the user interface of the MR device.

7. The method as claimed in claim 1, wherein the at least one application is configured to be scanner-independent.

8. The method as claimed in claim 1, wherein, to execute the at least one application, the at least one application is configured to provide control information to control at least one hardware component of the MR device using the control channel of the interface.

9. The method as claimed in claim 1, further comprising: prior to execution of the at least one application and/or during execution of the at least one application, providing dynamic control information relating to the MR device to the at least one application using the information channel of the interface.

10. The method as claimed in claim 1, wherein raw data of a magnetic resonance measurement is provided to the at least one application, using the information channel of the interface, for reconstruction.

11. The method as claimed in claim 1, wherein reconstructed magnetic resonance image data is provided by the at least one application using information channel of the interface.

12. The method as claimed in claim 1, wherein the interface is disposed directly on a system controller of the MR device and/or is connected to the system controller of via a network.

13. The method as claimed in claim 1, wherein the interface comprises a license module.

14. The method as claimed in claim 1, wherein the interface comprises a learning module having an artificial neural network.

15. A non-transitory computer-readable storage medium with an executable program stored thereon, that when executed, instructs a processor to perform the method of claim 1.

16. A magnetic resonance (MR) device comprising:
an interface; and
a controller configured to:
perform a communication and/or data exchange, using the interface, with at least one application stored on separately implemented memory;
communicate at least one item of hardware information relating to the MR device to the at least one application using the interface;
match at least one parameter of the at least one application to the at least one item of hardware information; and
execute the at least one application based on at least one item of control information provided by the at least one application.

17. A system comprising the MR device as claimed in claim 16 and at least one application.
